(19) United States
(12) Patent Application Publication
(10) Pub. No.: US 2019/0290558 A1
(43) Pub. Date: Sep. 26, 2019

(54) EMULSIFIED MAKEUP COSMETIC COMPOSITION WITH EXCELLENT SUSTAINABILITY AND METHOD FOR EVALUATING SUSTAINING FORCE FOR COSMETIC COMPOSITION

(71) Applicant: Amorepacific Corporation, Seoul (KR)

(72) Inventors: Hyunwol KANG, Yongin-si, Gyeonggi-do (KR); Yujin KANG, Yongin-si, Gyeonggi-do (KR); Kyung Nam KIM, Yongin-si, Gyeonggi-do (KR); Yeongjin CHOI, Yongin-si, Gyeonggi-do (KR)

(21) Appl. No.: 16/464,120

(22) PCT Filed: Nov. 27, 2017
(86) PCT No.: PCT/KR2017/013603

§ 371 Date: May 24, 2019

(30) Foreign Application Priority Data
Nov. 30, 2016 (KR) 10-2016-0162095

(51) Int. Cl. A61K 8/06; A61K 8/31; A61K 8/891; A61K 8/81

(52) U.S. Cl. CPC A61K 8/06; A61K 8/31; A61Q 1/00; A61K 8/8152; A61K 8/891

(57) ABSTRACT

Disclosed in the present specification is an emulsified makeup cosmetic composition with excellent sustainability. The composition has an effect of providing an emulsified makeup cosmetic composition wherein when the composition is coated on the skin using a volatile oil, the oil is evaporated and a film forming agent imparting high sustaining force is left, thereby preventing the smearing of the composition, increasing sustaining force, and improving the feeling of use even after drying. In addition, disclosed in the present specification is a method for evaluating sustaining force of the cosmetic composition.

### TECHNICAL FIELD

The present specification discloses an emulsified makeup cosmetic composition exhibiting excellent persistence and a method of evaluating the persistency of a cosmetic composition.

### BACKGROUND ART

In general, a water-in-oil type emulsified foundation has an oil outer phase. Thus it exhibits superior water resistance to an oil-in-water type product but it causes swelling and clumping of cosmetic film due to sweat. Hence, it is troublesome to frequently fix the makeup and there is a problem that the cosmetic leaves marks on the contacting object such as clothes since the cosmetic easily adheres to the clothes even by external contact. In addition, the cosmetic provides not only heavy and stiff feel of use but also a thick cosmetic film so that the breathability of the skin decreases, the skin hardly breathes, and the skin is frustrated.

Conventionally, film formers, oil-absorbing powders, and the like have been used in order to improve the disadvantages of such an emulsified foundation and to impart high persistence thereto, but the practical effect is negligible, and the use of non-volatile oil has caused stickiness and transfer phenomenon. Furthermore, a makeup cosmetic composition which is composed of a film former and a volatile oil on a continuous phase, and does not cause transfer as a film is obtained while the oil evaporates has a problem that the feel of use is stuffy and an uncomfortable film is formed after the volatile component evaporates. Meanwhile, a composition containing a styrene-ethylene-propylene copolymer or a copolymer of a monoolefin-based unsaturated carboxylic acid monomer and a fatty-chain acrylic ester monomer and the like have been proposed in order to improve this problem, but it is difficult to expect a satisfactory persistency improving effect, the feel of use is stuffy and uncomfortable after drying, and a dry non-moisturizing film is often formed.

### CITATION LIST

### Patent Literature

[Patent Literature 1]

Korean Patent Publication No. 10-2014-0146375

### SUMMARY OF INVENTION

### Technical Problem

In an aspect, the present specification has been made in order to solve the problems of conventional emulsified makeup cosmetic compositions as described above, and an object thereof is to provide an emulsified makeup cosmetic composition which uses a volatile oil so that the oil evaporates and a film former which imparts high persistency and exhibits hard surface properties is leaved when the composition is applied to the skin, thereby preventing transfer after being applied to the skin, exhibiting increased persistency, and exhibiting improved feel of use even after being dried.

In another aspect, an object of the present specification is to provide a method of evaluating the persistency of a cosmetic composition which can objectively evaluate the effect of preventing transfer of a cosmetic composition.

### Solution to Problem

In an aspect, the technique disclosed herein provides an emulsified makeup cosmetic composition exhibiting excellent persistence which contains a membrane former containing a carrier oil and a film former and in which the film former contains a silicone polymer or a silicone acrylate copolymer.

In an exemplary embodiment, the composition may not contain a non-volatile oil and an organic sunblock.

In an exemplary embodiment, a mixed weight of a dispersant and an emulsifier in the composition may be 6 wt % or less with respect to a total weight of the composition.

In an exemplary embodiment, a transfer prevention rate of the composition may be 70% or more, and the transfer prevention rate may be determined by conducting a transfer test of the composition by a method in which the composition is applied to artificial leather and dried at 55° C. for 3 hours and then vertical and horizontal frictional forces are simultaneously applied to the composition by rotating a contact member having a load of 2 kg by 720°, measuring an L value (brightness value) by using a colorimeter, and conducting calculation by the following formula:

transfer prevention rate (%)=dL_{b}/dLₐ×100

(In the above formula, dLₐ=Lₐ−L₀, dL_{b}=L_{b}−L₀, L₀=L value of the artificial leather before the composition is applied, Lₐ=L value of the artificial leather to which the composition is applied before the transfer test, and L_{b}=L value of the artificial leather to which the composition is applied after the transfer test).

In an exemplary embodiment, the carrier oil may be volatile.

In an exemplary embodiment, the carrier oil may be one or more selected from the group consisting of cyclopentasiloxane, isododecane, and dimethicone.

In an exemplary embodiment, the silicone polymer may be one or more selected from the group consisting of trimethylsiloxysilicate, polypropylsilsesquioxane, polymethylsilsesquioxane, dimethicone/vinyltrimethylsiloxysilicate crosspolymer, trimethylsiloxysilicate/dimethiconol crosspolymer, and trimethylsiloxysilylcarbamoyl pullulan.

In an exemplary embodiment, the silicone acrylate copolymer may be one or more selected from the group consisting of a butyl acrylate/hydroxypropyl dimethicone acrylate copolymer and an acrylate/polytrimethylsiloxymethacrylate copolymer.

In an exemplary embodiment, the carrier oil and the film former may be mixed at 40 to 90 wt % and 10 to 60 wt %, respectively, with respect to a total weight of the membrane former.

In an exemplary embodiment, the membrane former may be contained at 4 to 20 wt % with respect to a total weight of the composition.

In an exemplary embodiment, the film former may be contained at 1 to 8 wt % with respect to a total weight of the composition.

In an exemplary embodiment, the film former may be one or more of the following film formers:

i) trimethylsiloxysilicate having an MQ ratio of 0.7 or less;

ii) trimethylsiloxysilicate/dimethiconol crosspolymer;

iii) a film former containing trimethylsiloxysilicate and polypropylsilsesquioxane; and

iv) an acrylate/polytrimethylsiloxymethacrylate copolymer.

In an exemplary embodiment, trimethylsiloxysilicate and polypropylsilsesquioxane may be mixed in a mixed weight ratio of 6 to 7:3 to 4 in the iii).

In another aspect, the technique disclosed herein provides a method of evaluating persistency of a cosmetic composition, the method including:

applying an evaluation target material to artificial leather;

drying the evaluation target material and then applying friction to the evaluation target material by using a contact member to conduct a transfer test; and

measuring an L_{b} value (brightness value) of the artificial leather by using a colorimeter after the transfer test.

In an exemplary embodiment, the evaluation target material may contain a film former.

In an exemplary embodiment, the method may include measuring an Lₐ value (brightness value) of the artificial leather to which the evaluation target material is applied before the transfer test by using a colorimeter and comparing the Lₐ value with the L_{b} value.

In an exemplary embodiment, the persistency of the evaluation target material may be evaluated by measuring a transfer prevention rate by the following formula in the method:

transfer prevention rate (%)=dL_{b}/dLₐ×100

(In the above formula, dLₐ=Lₐ−L₀, dL_{b}=L_{b}−L₀, L₀=L value of the artificial leather before the evaluation target material is applied, Lₐ=L value of the artificial leather to which the evaluation target material is applied before the transfer test, and L_{b}=L value of the artificial leather to which the evaluation target material is applied after the transfer test).

In an exemplary embodiment, the drying may be conducted at 55° C. for 3 hours.

In an exemplary embodiment, vertical and horizontal frictional forces may be simultaneously applied to the evaluation target material by rotating a contact member having a load of 2 kg by 720° in the transfer test.

### Advantageous Effects of Invention

In an aspect, the present specification has been made in order to solve the problems of conventional emulsified makeup cosmetic compositions as described above and has an effect of providing an emulsified makeup cosmetic composition which uses a volatile oil so that the oil evaporates and a film former which imparts high persistency and exhibits hard surface properties is leaved when the composition is applied to the skin, thereby preventing transfer after being applied to the skin, exhibiting increased persistency, and exhibiting improved feel of use even after being dried.

In another aspect, the present specification has an effect of providing a method of evaluating the persistency of a cosmetic composition which can objectively evaluate the effect of preventing transfer of a cosmetic composition.

### BRIEF DESCRIPTION OF DRAWINGS

FIG. 1 illustrates a method of evaluating the persistency of a cosmetic composition according to a test example of the present invention.

FIG. 2 illustrates the results of comparative evaluation on the persistency of compositions through a transfer test of the compositions depending on the kind and content of film formers in a test example of the present specification.

### DESCRIPTION OF EMBODIMENTS

Hereinafter, the present invention will be described in detail.

In an aspect, the technique disclosed herein provides an emulsified makeup cosmetic composition exhibiting excellent persistence which contains a membrane former containing a carrier oil and a film former and in which the film former contains a silicone polymer or a silicone acrylate copolymer.

In an exemplary embodiment, the composition may not contain a non-volatile oil and an organic sunblock.

The emulsified makeup cosmetic composition disclosed herein does not contain a non-volatile oil or an organic sunblock such as octylmethoxycinnamate (OMC) or avobenzone and thus has an effect of preventing stickiness or transfer (smear) by external contact as compared with a conventional emulsified makeup cosmetic composition.

In an exemplary embodiment, the composition may not contain a dispersant. Alternatively, in another exemplary embodiment, the composition may contain a dispersant at 4 wt % or less, for example, 0.01 to 4 wt % or 0.01 to 3 wt % or 0.01 to 2 wt % or 0.01 to 1 wt % and an emulsifier at 6 wt % or less, for example, 0.01 to 6 wt % or 0.01 to 5 wt % or 0.01 to 4 wt % or 0.01 to 3 wt % or 0.01 to 2 wt % or 0.01 to 1 wt % with respect to the total weight of the composition, so that the contents of the dispersant and emulsifier may be lower than those in a conventional emulsified makeup cosmetic composition.

In an exemplary embodiment, the mixed weight of a dispersant and an emulsifier in the composition may be 6 wt % or less, for example, 0.01 to 6 wt % or 0.01 to 5 wt % or 0.01 to 4 wt % or 0.01 to 3 wt % or 0.01 to 2 wt % or 0.01 to 1 wt % with respect to the total weight of the composition.

In an exemplary embodiment, a transfer prevention rate of the composition may be 70% or more, and the transfer prevention rate may be determined by conducting a transfer test of the composition by a method in which the composition is applied to artificial leather and dried at 55° C. for 3 hours and then vertical and horizontal frictional forces are simultaneously applied to the composition by rotating a contact member having a load of 2 kg by 720°, measuring an L value (brightness value) by using a colorimeter, and conducting calculation by the following formula:

transfer prevention rate (%)=dL_{b}/dLₐ×100

(In the above formula, dLₐ=Lₐ−L₀, dL_{b}=L_{b}−L₀, L₀=L value of the artificial leather before the composition is applied, Lₐ=L value of the artificial leather to which the composition is applied before the transfer test, and L_{b}=L value of the artificial leather to which the composition is applied after the transfer test).

In an exemplary embodiment, the transfer prevention rate of the composition may be 70% or more, 75% or more, 80% or more, 85% or more, 90% or more, or 95% or more but 100% or less, 99% or less, 98% or less, 97% or less, 96% or less, or 95% or less.

In one exemplary embodiment, the composition may contain a volatile oil at 5 to 80 wt % with respect to the total weight of the composition. In addition, the content of the volatile oil may include the content of the carrier oil, and the volatile oil may be the same as or different from the carrier oil used in the membrane former. In another exemplary embodiment, the content of the volatile oil may be 5 wt % or more, 10 wt % or more, 15 wt % or more, 20 wt % or more, 25 wt % or more, 30 wt % or more, 35 wt % or more, 40 wt % or more, 45 wt % or more, 50 wt % or more, 55 wt % or more, 60 wt % or more, 65 wt % or more, 70 wt % or more, 75 wt % or more, or 80 wt % or more but 80 wt % or less, 75 wt % or less, 70 wt % or less, 65 wt % or less, 60 wt % or less, 55 wt % or less, 50 wt % or less, 45 wt % or less, 40 wt % or less, 35 wt % or less, 30 wt % or less, 25 wt % or less, 20 wt % or less, 15 wt % or less, 10 wt % or less, or 5 wt % or less.

In an exemplary embodiment, the volatile oil may include one or more selected from the group consisting of a silicone-based oil and a hydrocarbon-based oil.

In an exemplary embodiment, the volatile silicone-based oil may include one or more selected from the group consisting of cyclopentasiloxane, cyclohexasiloxane, cyclomethicone, phenyltrimethicone, methyltrimethicone, dimethicone, and trisiloxane.

In an exemplary embodiment, the dimethicone may have a viscosity of 2 cSt (centistokes) or less.

In an exemplary embodiment, the volatile hydrocarbon-based oil may include one or more selected from the group consisting of isododecane and dodecane.

In an exemplary embodiment, the carrier oil is volatile and thus is favorable for increasing the persistency of the makeup cosmetic composition and improving the feel of use thereof.

In an exemplary embodiment, the carrier oil may be one or more selected from the group consisting of cyclopentasiloxane, isododecane, and dimethicone.

In an exemplary embodiment, the dimethicone may have a viscosity of 2 cSt (centistokes) or less.

In an exemplary embodiment, the silicone polymer may be one or more selected from the group consisting of trimethylsiloxysilicate, polypropylsilsesquioxane, polymethylsilsesquioxane, dimethicone/vinyltrimethylsiloxysilicate crosspolymer, a trimethylsiloxysilicate/dimethiconol crosspolymer, and trimethylsiloxysilylcarbamoyl pullulan.

In an exemplary embodiment, the silicone acrylate copolymer may be one or more selected from the group consisting of a butyl acrylate/hydroxypropyl dimethicone acrylate copolymer and an acrylate/polytrimethylsiloxymethacrylate copolymer. The silicone acrylate copolymer provides persistence by acrylate polymer and soft feel of use by silicone.

In an exemplary embodiment, the carrier oil and the film former may be mixed at 40 to 90 wt % and 10 to 60 wt %, respectively, with respect to the total weight of the membrane former. In another exemplary embodiment, the carrier oil and film former may be mixed at 40 to 80 wt % and 20 to 60 wt %, or 40 to 70 wt % and 30 to 60 wt %, or 40 to 60 wt % and 40 to 60 wt % so that the persistency of the composition can be greatly increased.

In an exemplary embodiment, the content of the membrane former may be 4 to 20 wt % with respect to the total weight of the composition. In another exemplary embodiment, the content of the membrane former may be 4 wt % or more, 6 wt % or more, 8 wt % or more, 10 wt % or more, 12 wt % or more, 14 wt % or more, 16 wt % or more, 18 wt % or more, or 20 wt % or more but 20 wt % or less, 18 wt % or less, 16 wt % or less, 14 wt % or less, 12 wt % or less, 10 wt % or less, 8 wt % or less, 6 wt % or less, or 4 wt % or less.

In an exemplary embodiment, the film former may be contained at 1 to 8 wt % with respect to the total weight of the composition. An intended effect can be achieved by containing the film former at 1 wt % or more to increase the moisturizing ability and cosmetic persistency and containing the film former at 8 wt % or less to prevent stickiness, skin irritation, or skin stiffness in this manner.

From the above point of view, the film former may be mixed at 1 wt % or more, 3 wt % or more, 5 wt % or more, or 7 wt % or more but 8 wt % or less, 6 wt % or less, 4 wt % or less, or 2 wt % or less with respect to the total weight of the composition so that the persistency of the composition can be greatly increased.

The weight of the film former herein means the content based on the solids.

In an exemplary embodiment, the film former may be one or more of the following film formers.

i) trimethylsiloxysilicate having an MQ ratio of 0.7 or less;

ii) trimethylsiloxysilicate/dimethiconol crosspolymer;

iii) a film former containing trimethylsiloxysilicate and polypropylsilsesquioxane; and

iv) an acrylate/polytrimethylsiloxymethacrylate copolymer.

The film former described above does not form a conventional film which provides frustrating feel of use and has low persistency but forms a flexible film which does not cause a feel by foreign matter to the skin, and there is thus an effect of providing a makeup cosmetic composition which exhibits favorable spreadability, skin adhesion, water resistance, and smoothness and does not easily adhere by external contact.

In an exemplary embodiment, the carrier oil in the composition containing the film former described above may be cyclopentasiloxane or isododecane.

In an exemplary embodiment, the transfer prevention rate of the composition containing the film former described above may be higher than that of the composition containing isododecane as the carrier oil and trimethylsiloxysilicate having a MQ ratio of 0.8 as the film former, and the transfer prevention rate may be determined by conducting a transfer test of the composition by a method in which the composition is applied to the artificial leather and dried and a frictional force is applied to the composition by using a contact member, measuring the L value (brightness value) by using a colorimeter, and conducting calculating by the following formula:

transfer prevention rate (%)=dL_{b}/dLₐ×100

(In the above formula, dLₐ=Lₐ−L₀, dL_{b}=L_{b}−L₀, L₀=L value of the artificial leather before the composition is applied, Lₐ=L value of the artificial leather to which the composition is applied before the transfer test, and L_{b}=L value of the artificial leather to which the composition is applied after the transfer test).

In another exemplary embodiment, the transfer prevention rate of the composition containing the film former described above may be higher than that of the composition containing cyclopentasiloxane as the carrier oil and polymethylsilsesquioxane as the film former.

In an exemplary embodiment, the drying in the transfer test may be conducted at 55° C. for 3 hours.

In an exemplary embodiment, vertical and horizontal frictional forces may be simultaneously applied to the composition by rotating a contact member having a load of 2 kg by 720° in the transfer test.

In an exemplary embodiment, the contact surface of the contact member may be a muscle tape.

In the present specification, the MQ ratio means a ratio of a/b in the unit of M represented by (R¹₃SiO_{1/2}). and the unit of Q represented by (SiO_{4/2})_{b}, R¹ may be independently an alkyl group having 1 to 8 carbon atoms or an aryl group, and a and b represent integers greater than zero. Examples of the alkyl group may include methyl, ethyl, propyl, butyl, pentyl and hexyl, and examples of the aryl group may include phenyl, naphthyl, benzyl, tolyl, xylyl, and xenyl.

In an exemplary embodiment, the MQ ratio in the i) may be 0.5 to 0.7, specifically 0.7.

In an exemplary embodiment, a superior effect of improving the persistency of the composition can be obtained when trimethylsiloxysilicate and polypropylsilsesquioxane are mixed in a mixed weight ratio of 6 to 7:3 to 4 in the iii).

In an exemplary embodiment, it may be effective in preventing the transfer of the emulsified makeup cosmetic composition and improving the feel of use when the composition contains a pigment at 0 to 40 wt % with respect to the total weight of the composition. From this point of view, the composition may contain a pigment at 1 wt % or more, 5 wt % or more, 10 wt % or more, 15 wt % or more, 20 wt % or more but 40 wt % or less, 35 wt % or less, 30 wt % or less, 25 wt % or less, 20 wt % or less, 15 wt % or less, or 10 wt % or less with respect to the total weight of the composition.

In an exemplary embodiment, the pigment may be one or more selected from the group consisting of titanium oxide (titanium dioxide TiO₂), micro titanium oxide (micro TiO₂), zinc oxide (ZnO), micro zinc oxide (micro ZnO), iron oxide (FeO(II), Fe₂O₃(III), Fe₃O₄(IV)), aluminum oxide (alumina, Al₂O₃), barium sulfate (BaSO₃), silicon dioxide (silica, SiO₂), talc, kaolin, sericite, and mica, and the pigment may include those obtained by subjecting these to a surface treatment using an organic substance, an inorganic substance, or a silicone.

In an exemplary embodiment, the composition may contain components to be contained in conventional makeup compositions such as thickeners, pigments, nutrients, volatile solubilizers, antioxidants, preservatives, and fragrances to the extent to which the effect disclosed herein is not impaired, and these auxiliary components may be contained in an amount of 0 to 10 wt % with respect to the total weight of the composition.

In an exemplary embodiment, the composition may be a W/O or W/S formulation.

In an exemplary embodiment, the composition is not particularly limited as long as it is a liquid makeup cosmetic composition, and for example, the composition may be formulated into a sunblock, a liquid foundation, a makeup base, a skin cover, a concealer, and the like.

When the makeup cosmetic composition disclosed herein is applied to the skin, the volatile oil and the carrier oil volatilize. Accordingly, not only stickiness or transfer phenomenon after makeup is prevented but also the persisting membrane film performance that the composition is hardly removed after a long time has elapsed and the composition is dried is maintained since the film former described above is used, and an effect of providing excellent feel of use such as softness, moisturizing property, and comfort is provided.

In another aspect, the technique disclosed herein provides a method of quantitatively evaluating the persistency of a cosmetic composition. The method includes applying an evaluation target material to artificial leather; drying the evaluation target material and then applying friction to the evaluation target material by using a contact member to conduct a transfer test; and measuring an L_{b} value (brightness value) of the artificial leather by using a colorimeter after the transfer test.

In an exemplary embodiment, the evaluation target material may contain a film former.

In an exemplary embodiment, the evaluation target material may be an emulsified makeup cosmetic composition.

In an exemplary embodiment, the method may include measuring Lₐ value (brightness value) of artificial leather to which the evaluation target material is applied before the transfer test by a colorimeter and comparing the Lₐ value with the L_{b} value. The Lₐ value can be measured after the evaluation target material is applied to artificial leather or after the evaluation target material is applied to artificial leather and then dried.

In an exemplary embodiment, the persistency of the evaluation target material may be evaluated by measuring the transfer prevention rate by the following formula in the method:

transfer prevention rate (%)=dL_{b}/dLₐ×100

(In the above formula, dLₐ=Lₐ−L₀, dL_{b}=L_{b}−L₀, L₀=L value of the artificial leather before the evaluation target material is applied, Lₐ=L value of the artificial leather to which the evaluation target material is applied before the transfer test, and L_{b}=L value of the artificial leather to which the evaluation target material is applied after the transfer test).

In an exemplary embodiment, it may be evaluated that the persistency is high as the transfer prevention rate is high in the method.

In an exemplary embodiment, it may be evaluated that the persistency is high when the transfer prevention rate is 80% to 100% in the method.

In an exemplary embodiment, the drying may be conducted at 55° C. for 3 hours.

In an exemplary embodiment, vertical and horizontal frictional forces may be simultaneously applied to the evaluation target material by rotating a contact member having a load of 2 kg by 720° in the transfer test.

In an exemplary embodiment, the contact surface of the contact member may be a muscle tape.

### EMBODIMENTS

Hereinafter, the present invention will be described in more detail with reference to Examples. It should be apparent to those skilled in the art that these Examples are for illustrative purposes only and that the scope of the present invention is not construed as being limited by these Examples.

### Test Example 1. Preparation of Foundation Formulation

The composition of the membrane former which imparts persistency to the makeup cosmetic composition is presented in the following Table 1, and the specific kinds and contents of the carrier oils and the film formers are presented with respect to 100% of the total mixed weight thereof.

In addition, foundation formulations were prepared according to the composition presented in the following Table 2 by using the membrane formers of Examples 1 to 16 composed of the carrier oils and the film formers presented in Table 1 according to a conventional method. In the foundation formulations, the same raw materials were used for the components other than the membrane formers. The foundation formulations were prepared by setting the contents of the membrane formers of Examples 1 to 16 contained in the foundation formulations to 4%, 8%, 12%, 16%, and 20%, respectively.

TABLE 1

| | Carrier oil (wt %) | Film former (wt %) |
|---|---|---|
| Example 1 | Cyclopentasiloxane (50%) | Trimethylsiloxysilicate |
| | | (MQ ratio 0.8, 50%) |
| Example 2 | Cyclopentasiloxane (50%) | Polypropylsilsesquioxane (50%) |
| Example 3 | Cyclopentasiloxane (50%) | Trimethylsiloxysilicate |
| | | (MQ ratio 0.8, 25%) and polypropylsilsesquioxane (25%) |
| Example 4 | Cyclopentasiloxane (50%) | Polymethylsilsesquioxane (50%) |
| Example 5 | Cyclopentasiloxane (50%) | Trimethylsiloxysilicate |
| | | (MQ ratio 0.8, 31.5%) and polypropylsilsesquioxane (17.5%) |
| Example 6 | Isododecane (40%) | Trimethylsiloxysilicate (MQ ratio 0.8, 60%) |
| Example 7 | Cyclopentasiloxane (50%) | Trimethylsiloxysilicate (MQ ratio 0.7, 50%) |
| Example 8 | Isododecane (60%) | Butyl acrylate/hydroxypropyldimethicone acrylate |
| | | copolymer (40%) |
| Example 9 | Isododecane (60%) | Acrylate/polytrimethylsiloxymethacrylate copolymer (40%) |
| (Tg 70 to 80° C.) | | |
| Example 10 | Cyclopentasiloxane (80%) | Dimethicone/vinyltrimethylsiloxysilicate crosspolymer (20%) |
| Example 11 | Cyclopentasiloxane (85%) | Dimethicone/vinyltrimethylsiloxysilicate crosspolymer (15%) |
| Example 12 | Cyclopentasiloxane (70%) | Acrylate/polytrimethylsiloxymethacrylate copolymer (30%) |
| (Tg 70 to 80° C.) | | |
| Example 13 | Dimethicone (2 cSt, 60%) | Acrylate/polytrimethylsiloxymethacrylate copolymer (40%) |
| (Tg 45 to 60° C.) | | |
| Example 14 | Cyclopentasiloxane (60%) | Trimethylsiloxysilicate/dimethiconol crosspolymer (40%) |
| Example 15 | Isododecane (60%) | Trimethylsiloxysilicate/dimethiconol crosspolymer (40%) |
| Example 16 | Isododecane (70%) | Trimethylsiloxysilylcarbamoyl pullulan (30%) |

TABLE 2

| | Content (wt %) |
|---|---|
| Membrane formers of Examples 1 to 16 | 4 to 20 |
| Volatile oil | To 100 |
| Emulsifier and dispersant | 2 to 6 |
| Thickener | 0 to 1 |
| Water | 10 to 40 |
| Salt | 0 to 1 |
| Polyol and preservative | 0.5 to 6 |
| Pigment | 13 |
| Micro titanium dioxide | 4 to 5 |
| Total | 100 |

### Test Example 2. Evaluation on Transfer (Smear)

The evaluation on transfer of the foundation formulations prepared according to the composition presented in Table 2 was conducted as follows.

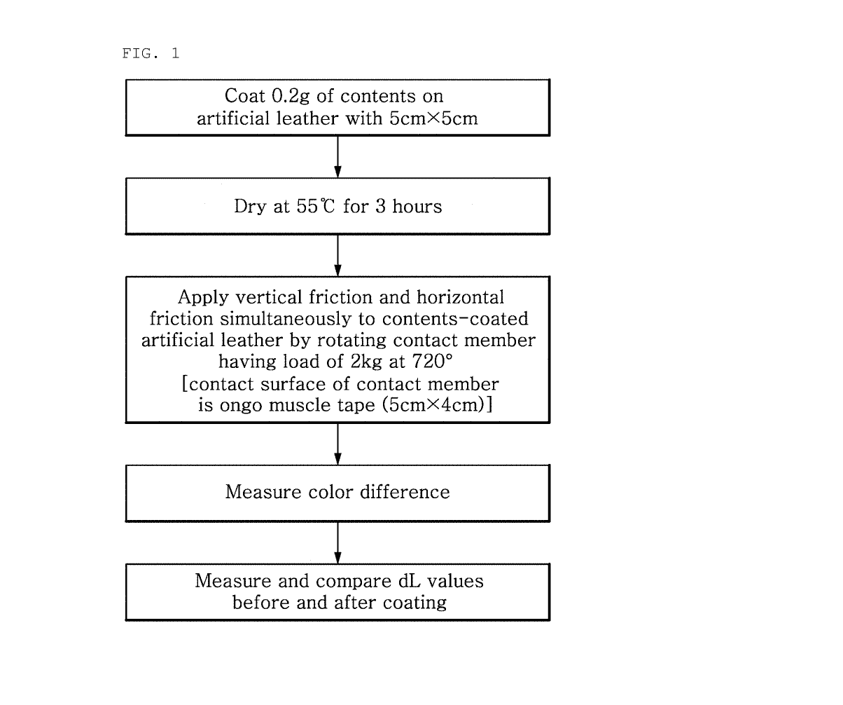

Specifically, the evaluation on transfer was conducted by a method in which the foundation formulation was applied to artificial leather in an amount larger than the amount to be actually applied to the skin, and dried at 55° C. for 3 hours, and vertical and horizontal frictional forces were applied to the foundation formulation by rotating a contact member having a load of 2 kg by 360° two times (see FIG. 1). A muscle tape (Kinesiology tape, ongo) was used as the contact surface of the contact member, the color difference before and after transfer was measured by using a colorimeter (KONICA MINOLTA spectrophotometer, CM-5), and the L value of the artificial leather was measured to quantify the extent of transfer. The L value was measured three times by repeatedly conducting the same experiment and expressed as the average value thereof.

The transfer prevention rate (%), namely, the residual amount (%) of the composition, was measured through the evaluation on transfer described above and presented in the following Table 3.

transfer prevention rate (%)=dL_{b}/dLₐ×100
dLₐ=Lₐ−L₀,
dL_{b}=L_{b}−L₀,

L₀=L value of artificial leather before composition is applied,

Lₐ=L value of artificial leather to which composition is applied before transfer test, and

L_{b}=L value of artificial leather to which composition is applied after transfer test.

TABLE 3

| Before (std. L = | | After (std. L = | | |
|---|---|---|---|---|
| 30.31) | | 30.31) | | Residual |
| L | dL | L | dL | amount % |

| | | | | | |
|---|---|---|---|---|---|
| Foundation of A | 65.8 | 35.49 | 46.31 | 16 | 45.08312 |
| company | | | | | |
| Foundation of B | 66.62 | 36.31 | 49.51 | 19.2 | 52.878 |
| company | | | | | |
| Foundation of C | 71.14 | 40.82 | 48.4 | 18.09 | 44.31651 |
| company | | | | | |

| | | | | | | |
|---|---|---|---|---|---|---|
| Example 1 | 4% | 65.9 | 35.59 | 50.7 | 20.39 | 57.29137 |
| | 8% | 67.3 | 36.99 | 51.99 | 21.68 | 58.61044 |
| | 12% | 69.07 | 38.75 | 57.23 | 26.92 | 69.47097 |
| | 16% | 70.4 | 40.09 | 60.45 | 30.14 | 75.18084 |
| | 20% | 70.63 | 40.32 | 62.44 | 32.13 | 79.6875 |
| Example 2 | 4% | 64.58 | 34.26 | 52.26 | 21.95 | 64.06888 |
| | 8% | 65.76 | 35.45 | 50.72 | 20.41 | 57.57405 |
| | 12% | 68.46 | 38.15 | 52.95 | 22.64 | 59.34469 |
| | 16% | 69.96 | 39.65 | 59.87 | 29.56 | 74.55233 |
| | 20% | 70.61 | 40.3 | 59.93 | 29.61 | 73.47395 |
| Example 3 | 4% | 63.98 | 33.66 | 52.36 | 22.05 | 65.50802 |
| | 8% | 65.95 | 35.63 | 53.53 | 23.22 | 65.1698 |
| | 12% | 68.55 | 38.24 | 57.81 | 27.49 | 71.88808 |
| | 16% | 69.22 | 38.91 | 55.98 | 25.67 | 65.97276 |
| Example 4 | 4% | 65.58 | 35.27 | 54.1 | 23.79 | 67.45109 |
| | 8% | 68.02 | 37.7 | 52.32 | 22 | 58.35544 |
| Example 5 | 4% | 65.63 | 35.32 | 49.91 | 19.59 | 55.46433 |
| | 8% | 68.11 | 37.79 | 54.89 | 24.58 | 65.04366 |
| | 12% | 69.18 | 38.86 | 56.92 | 26.61 | 68.47658 |
| | 16% | 69.75 | 39.44 | 61.83 | 31.52 | 79.91886 |
| | 20% | 70.37 | 40.05 | 65.53 | 35.22 | 87.94007 |
| Example 6 | 12% | 69.88 | 39.57 | 56.26 | 25.95 | 65.57998 |
| Example 7 | 12% | 68.44 | 38.12 | 63.54 | 33.23 | 87.17209 |
| Example 8 | 4% | 65.58 | 35.26 | 53.1 | 22.79 | 64.63415 |
| | 8% | 67.53 | 37.22 | 56.3 | 25.98 | 69.80118 |
| | 12% | 68.49 | 38.17 | 57.88 | 27.57 | 72.2295 |
| | 16% | 67.82 | 37.5 | 56.02 | 25.71 | 68.56 |
| | 20% | 66.72 | 36.4 | 57.82 | 27.5 | 75.54945 |
| Example 9 | 4% | 65.96 | 35.64 | 60.97 | 30.66 | 86.02694 |
| | 8% | 67.54 | 37.23 | 65.57 | 35.26 | 94.70857 |
| | 12% | 69.11 | 38.8 | 68.98 | 38.66 | 99.63918 |
| | 16% | 68.98 | 38.67 | 68.87 | 38.56 | 99.71554 |
| | 20% | 68.81 | 38.49 | 69.11 | 38.8 | 100.8054 |
| Example 10 | 8% | 64.37 | 34.06 | 54.26 | 23.95 | 70.31709 |
| | 16% | 64.64 | 34.32 | 56.58 | 26.27 | 76.54429 |
| Example 11 | 8% | 65.32 | 35.01 | 55.1 | 24.79 | 70.80834 |
| | 16% | 65.38 | 35.06 | 57.14 | 26.82 | 76.49743 |
| Example 12 | 16% | 68.02 | 37.71 | 67.51 | 37.19 | 98.62106 |
| Example 13 | 12% | 67.33 | 37.01 | 59.65 | 29.34 | 79.27587 |
| Example 14 | 12% | 68.59 | 38.28 | 62.32 | 32.01 | 83.62069 |
| Example 15 | 12% | 68.83 | 38.52 | 61.69 | 31.38 | 81.46417 |
| Example 16 | 4% | 66.16 | 36 | 57.53 | 27.36 | 76 |

As a result, unlike the conventional foundation formulations, the foundation formulations containing the membrane formers of Examples 1 to 16 exhibited a transfer prevention rate (residual amount) of 60% or more and provided excellent feel of use such as spreadability, skin adhesion, softness, moisturizing property, and comfort.

Example 9 exhibited a high transfer preventing effect of 90% or more at a content of 8% or more and exhibited a significantly high transfer preventing effect of 99% or more at a content of 12% (containing a film former at 4.8% based on solids) or more.

Meanwhile, Example 12 (16%) exhibited a lower transfer preventing effect than Example 9 (12%) when the content of solids was the same, and it was thus found that the strength of cosmetic film varied depending on the carrier oil even when the components and the contents of solids thereof were the same as each other. Example 12 exhibited a high transfer preventing effect of 90% or more at a content of 16% or more.

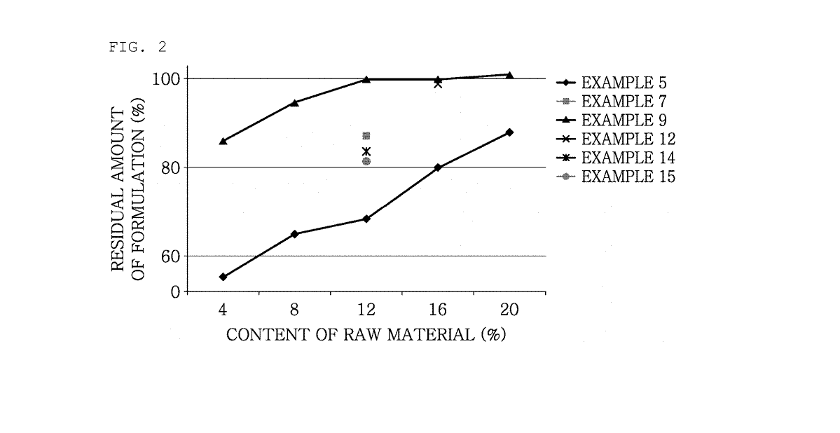

In addition, Example 5 exhibited a high transfer preventing effect of 80% or more at a content of 20% or more, and it was found that the effect was different depending on the ratio of trimethylsiloxysilicate to polypropylsilsesquioxane when being compared with Example 3. Example 7 exhibited a high transfer preventing effect of 80% or more at a content of 12% or more and it was thus confirmed that a specific MQ ratio affects the transfer preventing effect. Example 9 exhibited a high transfer preventing effect of 80% or more at a content of 4% or more and Example 14 and Example 15 exhibited a high transfer preventing effect of 80% or more at a content of 12% or more (see FIG. 2).

Specific portions of the present invention have been described in detail, those skilled in the art will appreciate that these specific embodiments are merely preferred embodiments and that the scope of the present invention is not limited thereby. It is thus intended that the scope of the present invention be defined by the appended claims and their equivalents.

1. An emulsified makeup cosmetic composition exhibiting excellent persistence comprising a membrane former containing a carrier oil and a film former, wherein the film former contains a silicone polymer or a silicone acrylate copolymer.

2. The composition according to claim 1, wherein the composition does not comprise a non-volatile oil and an organic sunblock.

3. The composition according to claim 1, wherein a mixed weight of a dispersant and an emulsifier is 6 wt % or less with respect to a total weight of the composition.

4. The composition according to claim 1, wherein
a transfer prevention rate of the composition is 70% or more, and
the transfer prevention rate is determined by conducting a transfer test of the composition by a method in which the composition is applied to artificial leather and dried at 55° C. for 3 hours and then vertical and horizontal frictional forces are simultaneously applied to the composition by rotating a contact member having a load of 2 kg by 720°, measuring an L value (brightness value) by using a colorimeter, and conducting calculation by the following formula:
transfer prevention rate (%)=dL_{b}/dLₐ×100
wherein in the above formula,
dLₐ=Lₐ−L₀,
dL_{b}=L_{b}−L₀,
L₀=L value of the artificial leather before the composition is applied,
Lₐ=L value of the artificial leather to which the composition is applied before the transfer test, and
L_{b}=L value of the artificial leather to which the composition is applied after the transfer test.

5. The composition according to claim 1, wherein the carrier oil is volatile.

6. The composition according to claim 1, wherein the carrier oil is one or more selected from the group consisting of cyclopentasiloxane, isododecane, and dimethicone.

7. The composition according to claim 1, wherein the silicone polymer is one or more selected from the group consisting of trimethylsiloxysilicate, polypropylsilsesquioxane, polymethylsilsesquioxane, dimethicone/vinyltrimethylsiloxysilicate crosspolymer, trimethylsiloxysilicate/dimethiconol crosspolymer, and trimethylsiloxysilylcarbamoyl pullulan.

8. The composition according to claim 1, wherein the silicone acrylate copolymer is one or more selected from the group consisting of a butyl acrylate/hydroxypropyl dimethicone acrylate copolymer and an acrylate/polytrimethylsiloxymethacrylate copolymer.

9. The composition according to claim 1, wherein the carrier oil and the film former are mixed at 40 to 90 wt % and 10 to 60 wt %, respectively, with respect to a total weight of the membrane former.

10. The composition according to claim 1, wherein the membrane former is contained at 4 to 20 wt % with respect to a total weight of the composition.

11. The composition according to claim 1, wherein the film former is contained at 1 to 8 wt % with respect to a total weight of the composition.

12. The composition according to claim 1, wherein the film former is one or more of the following film formers:
i) trimethylsiloxysilicate having an MQ ratio of 0.7 or less;
ii) trimethylsiloxysilicate/dimethiconol crosspolymer;
iii) a film former containing trimethylsiloxysilicate and polypropylsilsesquioxane; and
iv) an acrylate/polytrimethylsiloxymethacrylate copolymer.

13. The composition according to claim 12, wherein the trimethylsiloxysilicate and polypropylsilsesquioxane are mixed in a mixed weight ratio of 6 to 7:3 to 4 in the iii).

14. A method of evaluating persistency of a cosmetic composition, the method comprising:
applying an evaluation target material to artificial leather;
drying the evaluation target material and then applying friction to the evaluation target material by using a contact member to conduct a transfer test; and
measuring an L_{b} value (brightness value) of the artificial leather by using a colorimeter after the transfer test.

15. The method of evaluating persistency of a cosmetic composition according to claim 14, wherein the evaluation target material contains a film former.

16. The method of evaluating persistency of a cosmetic composition according to claim 14, comprising:
measuring an Lₐ value (brightness value) of the artificial leather to which the evaluation target material is applied before the transfer test by using a colorimeter and comparing the Lₐ value with the L_{b} value.

17. The method of evaluating persistency of a cosmetic composition according to claim 14, wherein the persistency of the evaluation target material is evaluated by measuring a transfer prevention rate by the following formula:
transfer prevention rate (%)=dL_{b}/dLₐ×100
wherein in the above formula,
dLₐ=Lₐ−L₀,
dL_{b}=L_{b}−L₀,
L₀=L value of the artificial leather before the evaluation target material is applied,
Lₐ=L value of the artificial leather to which the evaluation target material is applied before the transfer test, and
L_{b}=L value of the artificial leather to which the evaluation target material is applied after the transfer test.

18. The method of evaluating persistency of a cosmetic composition according to claim 14, wherein the drying is conducted at 55° C. for 3 hours.

19. The method of evaluating persistency of a cosmetic composition according to claim 14, wherein vertical and horizontal frictional forces are simultaneously applied to the evaluation target material by rotating a contact member having a load of 2 kg by 720° in the transfer test.

20. A method for improving persistence of an emulsified makeup cosmetic composition, comprising adding a membrane former containing a carrier oil and a film former to form an emulsified makeup cosmetic composition, wherein the film former contains a silicone polymer or a silicone acrylate copolymer.
